(19) United States
(12) Patent Application Publication
(10) Pub. No.: US 2012/0059165 A1
(43) Pub. Date: Mar. 8, 2012

(54) INTERMEDIATE COMPOUND FOR SYNTHESIZING PHARMACEUTICAL AGENT AND PRODUCTION METHOD THEREOF

(75) Inventors: Rikizou Furuya, Osaka (JP); Hiroki Ozawa, Osaka (JP); Eiji Toyofuku, Osaka (JP); Shinichi Kusaka, Osaka (JP); Hiroshi Iwamura, Osaka (JP); Daiki Sakai, Osaka (JP); Kazuki Nakayama, Osaka (JP); Kazutoshi Watanabe, Osaka (JP)

(73) Assignee: SANOFI, Paris (FR)

(21) Appl. No.: 13/255,748

(22) PCT Filed: Mar. 9, 2010
(86) PCT No.: PCT/JP2010/054285

§ 371 Date: Nov. 18, 2011

Related U.S. Application Data
(60) Provisional application No. 61/158,798, filed on Mar. 10, 2009.

(51) Int. Cl. C07D 413/10; C07D 413/14

(52) U.S. Cl. 544/123; 544/138

(57) ABSTRACT

A production method of an optically active 2-{4-(5-substituted-oxadiazolyl) phenyl}morpholine which is useful as an intermediate for synthesizing a pharmaceutical agent is provided and the method comprises the following steps 1) to 4): 1) reacting a bromophenylmorpholine with a hexacyanoferrate(II) or a hydrate thereof at a temperature of from 110° C. to 140° C. in a reaction mixture comprising a Na₂CO₃, an organophosphorus compound, and a palladium catalyst in a polar aprotic solvent alone or combination of a polar aprotic solvent and other polar aprotic solvent or hydrocarbon solvent to give a cyanophenylmorpholine; 2) reacting the cyanophenylmorpholine with hydroxylamine or hydroxylamine hydrochloride at a temperature of from 10° C. to 40° C. in an aprotic polar solvent to give a hydroxylamine derivative; 3) reacting the hydroxylamine derivative with an acylation reagent selected from the group consisting of aliphatic acyl halides, aromatic acyl halides, aliphatic acyl anhydrides and aromatic anhydrides; and 4) keeping the mixture obtained after step 3) at a temperature of from 60° C. to 140° C. to give a 2-{4-(5-substituted-oxadiazolyl) phenyl}morpholine.

### TECHNICAL FIELD

The present invention relates to a production method of an optically active 2-[4-(5-substituted-oxadiazoly)phenyl]morpholine and a novel compound obtained by the production method. The compound produced according to the present invention is useful as an intermediate for synthesizing a pharmaceutical agent.

### BACKGROUND ART

A compound such as 2-(2-arylmorpholin-4-yl)-1-methyl-1H-[4,4′]bipyrimidinyl-6-one represented by the following formula (i) or the like has tau protein kinase 1 inhibitory action and is useful as a therapeutic drug for Alzheimer' s disease and the like, as disclosed in WO 2009/035162. The patent document also discloses that the compound represented by formula (i) is produced from the compound represented by formula (iii) and the morpholine compound represented by formula (ii) as starting materials.

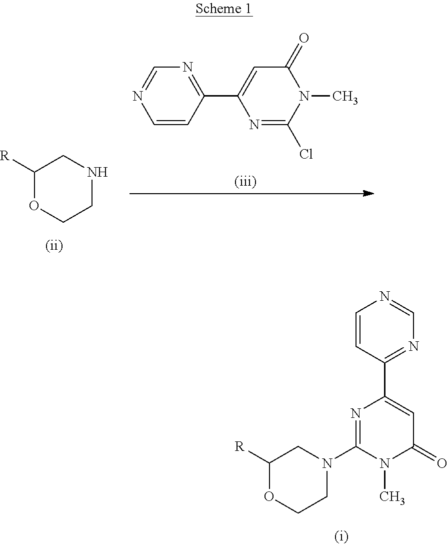

(In Scheme 1, R represents a benzene ring which may be substituted.)

Therefore, the morpholine compound represented by formula (ii) is used as an intermediate for synthesizing a pharmaceutical agent.

The production method of one of the compounds represented by formula (ii), 2-(4-(5-methyloxadiazolyl)phenyl)morpholine, is disclosed in WO 2009/035162 and WO2008/078837. The method includes a reaction of 2-(4-bromophenyl)morpholine to produce 2-(4-formylphenyl)morpholine, and a reaction of 2-(4-formylphenyl)morpholine to produce 2-(4-cyanophenyl) morpholine. However, the reaction described in the documents for the synthesis of the cyanophenyl morpholine via the formylphenyl morpholine was not considered to be suitable for industrial scale production because the reaction progresses under an ultra low temperature.

WO 99/02525 discloses a production method of oxadiazolylphenyl-oxazolidinones, wherein an oxadiazolylphenyl compound can be obtained from a cyanophenyl compound by the ring formation with an organic acid anhydride. The aminophenyl-oxazolidinone is converted into the cyanophenyl compound via a diazophenyl-oxazolidinone prior to the above reaction. However, the above method, which includes formation of a diazo compound, is considered to be undesirable as industrial production. In addition, the production method of the optically active morpholine compound having cyano-substituted aryl group as a substituent is not described in WO 99/02525.

Chem. Commun., 2004, 1388-1389 and U.S. 2006/0106223 disclose a production method of substituted benzonitriles using potassium hexacyanoferrate(II). However, the result concerning the reaction to a compound that contains morpholine unit in its structure is not included in either reference.

### Citation List

### Patent Literature

WO 2009/035162

WO 2008/078837

WO 99/02525

U.S. 2006/0106223

### Non Patent Literature

Chem. Commun., 2004, 1388-1389.

### SUMMARY OF INVENTION

### Technical Problem

It is therefore an object of the present invention to provide a novel and efficient production method of optically active 2-[4-(5-substituted-oxadiazolyl)phenyl]morpholines and a novel compound obtained thereby.

### Solution to Problem

The inventors of the present invention have conducted intensive studies in an attempt to solve the aforementioned problems and found an efficient production method of optically active 2-[4-(5-substituted-oxadiazolyl)phenyl]morpholines via an optically active 2-(4-bromophenyl)morpholine, which resulted in the completion of the present invention.

Accordingly, the present invention provides a production method of optically active 2-[4-(5-substituted-oxadiazolyl)phenyl]morpholines represented by formula 5, which comprises the following steps 1) to 4):

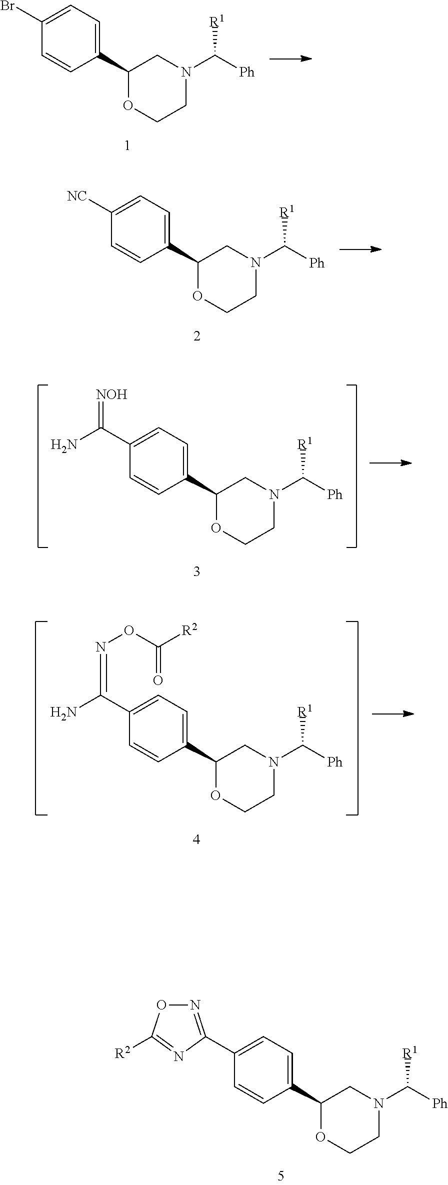

wherein R¹ represents hydrogen atom or a C₁-C₆ alkyl group, R² represents a C₁-C₆ alkyl group, a substituted C₁-C₆ alkyl group, or an aryl group,
1) reacting a compound represented by formula 1 with an alkali metal salt of hexacyanoferrate(II) or a hydrate thereof such as K₄[Fe(CN)₆]-3H₂O at a temperature of from 110° C. to 140° C. in a reaction mixture comprising Na₂CO₃, an organophosphorus compound selected from the group consisting of 2-dicyclohexylphosphino-2′,4′,6′-triisopropylbiphenyl, 1,1′-bis(diphenylphosphino)ferrocene, trio-toly)phosphine, and 1,3-bis(diphenylphosphino)propane, and a palladium catalyst (for example Pd(OAC)₂) in a polar aprotic solvent selected from the group consisting of N,N-dimethylacetoamide, N-methylpyrroridone, N,N-dimethylformamide, and tetrahydrofuran, or combination of an polar aprotic solvent and other polar aprotic solvent or hydrocarbon solvent (for example N,N-dimethylacetoamide-tolunene) to give a compound represented by formula 2,
2) reacting the compound represented by formula 2 with hydroxylamine or hydroxylamine hydrochloride at a temperature of from 10° C. to 40° C. in an aprotic polar solvent to give a compound represented by formula 3,
3) reacting the compound represented by formula 3 with an acylation reagent selected from the group consisting of aliphatic acyl halides, aromatic acyl halides, aliphatic acyl anhydrides and aromatic acyl anhydrides, preferably to give a compound represented by formula 4, and
4) keeping the mixture obtained after step 3) at a temperature of from 60° C. to 140° C., preferably to react the compound represented by formula 4 at a temperature of from 60° C. to 140° C., to give a compound represented by formula 5.

The present invention moreover provides a production method of an optically active 2-[4-(5-substituted-oxadiazolyl)phenyl]morpholines represented by formula 7, which comprises the following steps 5) and 6):

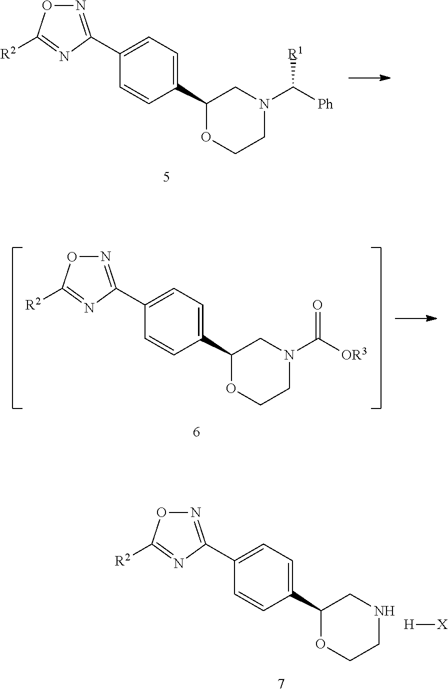

wherein R¹ represents hydrogen atom or a C₁-C₆ alkyl group; R² represents a C₁-C₆ alkyl group, a C₆-C₁₀ aryl group, or a 5- or 6-membered heteroaryl group; R³ represents a phenyl group, a benzyl group, or a fluorenylmethyl group; H—X represents an organic acid,
5) reacting the compound represented by formula 5 with a chloroformate selected from the group consisting of phenylchloroformate, benzylchloroformate, and fluorenylmethyloxycarbonylchloride in a solvent to give a compound represented by formula 6, and
6) subjecting the compound represented by formula 6 to hydrolysis or aminolysis, and reacting the resulting unprotected 2-[4-(5-substituted-oxadiazolyl)phenyl]morpholine with an organic acid in a polar solvent to give the compound represented by formula 7.

The present invention further provides an optically active 2-[4-(5-substituted-oxadiazoly)phenyl]morpholine represented by formula 8:

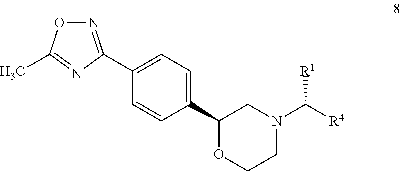

wherein R¹ represents hydrogen atom or a C₁-C₆ alkyl group; R⁴ represents a C₁-C₆ alkyl group or an aryl group optionally having substituent(s).

The present invention also provides an optically active compound represented by formula 9:

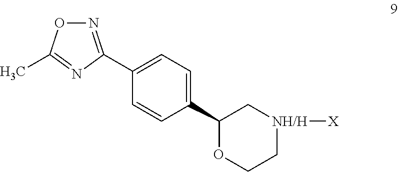

wherein H—X represents a C₁-C₅ alkyl carboxylic acid, a C₁-C₅ alkyl sulfonic acid, an aryl-carboxylic acid, or an aryl sulfonic acid.

The present invention further provides a production method of an optically active compound (I), which comprises the step of producing an intermediate in accordance with any one of the above-mentioned methods, preferably the step of producing an intermediate comprising the above steps 1) to 6) in this order.

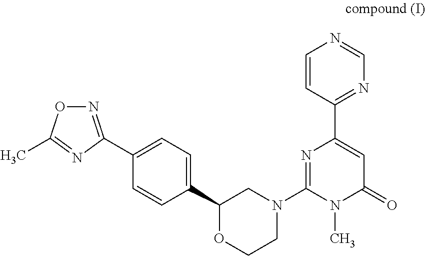

### Advantageous Effects of Invention

The optically active morpholine compound obtained by the production method of the present invention can be used as a starting material for synthesizing 2-(2-arylmorpholin-4-yl)-1-(R″)-1H-[4,4′]bipyrimidinyl-6-one, which is useful as a therapeutic drug for Alzheimer' s disease and the like, as shown in the following formula. In addition, the production method of the present invention gives good yield of the product, and is industrially advantageous.

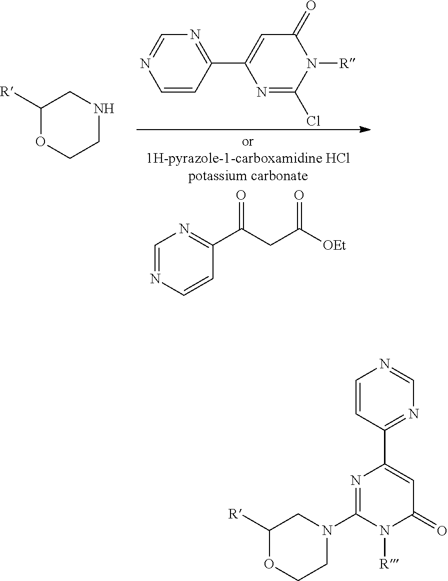

(In the above scheme, R′ represents a 4-(5-substituted-oxadiazolyl)phenyl group, R″ represents a C₁-C₁₂ alkyl group, R′″ represents hydrogen atom, or a C₁-C₁₂ alkyl group.)

### DESCRIPTION OF EMBODIMENTS

The invention is explained in detail as follows.

In this specification, the halogen atom may be chlorine, bromine, iodine, or fluorine atom.

In this specification, the C₁-C₆ alkyl group may be a straight chain or branched chain alkyl group having 1 to 6 carbon atoms. The examples of the C₁-C₆ alkyl group include methyl group, ethyl group, propyl group, isopropyl group, butyl group, secondary butyl group, tertiary butyl group, pentyl group, hexyl group and the like.

In this specification, the substituted C₁-C₆ alkyl group may be a C₁-C₆ alkyl group having 1 to 3 substituents selected from a halogen (chlorine, bromine, iodine, fluorine) atom, hydroxyl group, nitro group, an amino group, cyano group, a C₁-C₆ alkyl group, and a C₁-C₆ alkoxy group. The examples of the substituted C₁-C₆ alkyl group include chloromethyl group, methoxymethyl group and the like.

In this specification, the aryl group may be an aryl group having 6 to 10 carbon atoms. The examples of the aryl group include phenyl group, naphthyl group and the like.

In this specification, an alkali metal salt of hexacyanoferrate(II) is preferably a sodium or potassium salt of hexacyanoferrate(II) represented by Mₙ[Fe(CN)₆], wherein M represents Na or K, and n represents 3 or 4. Examples of the hexacyanoferrate(II) include K₄[Fe(CN)₆], Na₄[Fe(CN)₆] and K₃[Fe(CN)₆]. A hydrate of the salt of hexacyanoferrate(II) such as K₄[Fe(CN)₆]-3H₂O or K₄[Fe(CN)₆]-10H₂O may also be used.

In this specification, the organophosphorus compound may be a chemical compound containing a carbon-phosphorus bond. The examples of the organophosphorus compound include 2-dicyclohexylphosphino-2′,4′,6′-triisopropylbiphenyl, 1,1′-bis(diphenylphosphino)ferrocene, tri(o-tolyl)phosphine, and 1,3-Bis(diphenylphosphino)propane.

In this specification, the palladium catalyst may be a palladium salt. The examples of the palladium catalyst include palladium diacetate, palladium dichloride, tris(dibenzyldeneacetone)dipalladium and the like.

In this specification, the polar solvent may be a solvent that has electric bias as the solvent molecule. A solvent that has total dipole moment (sum of dipole moments of the molecules in the solvent) of zero may be included in the definition of polar solvent when the moment of the solvent is locally noticeable. The examples of such solvent include water and ethanol.

In this specification, the polar aprotic solvent is a solvent that shares ion dissolving power with protic solvents but lack an acidic hydrogen. A polar aprotic solvent generally has a high dielectric constant and high polarity. The examples of the polar aprotic solvent include dimethyl sulfoxide, dimethylformamide, dioxane hexamethylphosphorotriamide, tetrahydrofuran, N,N-dimethylacetoamide, N-methylpyrroridone, and the like.

In this specification, a hydrocarbon solvent is a solvent that consists of a liquid hydrocarbon at room temperature. The examples of the hydrocarbon solvent include benzene, kerosene, xylene, and other petroleum derivatives.

In this specification, an ether contain an ether group—an oxygen atom connected to two alkyl or aryl groups—of general formula. The examples of the ether include diethyl ether, cyclopentyl methyl ether, tetrahydrofuran, and t-butyl methyl ether.

In this specification, the hydroxylamine or its inorganic salt may be a chemical compound such as hydroxylamine free base in water, hydroxylamine hydrochloride, hydroxylamine sulfate and the like.

In this specification, the acylation reagent may be either of two common acylation agents, an acid halides and an anhydride, each of which is represented by a general formula RCO—X, wherein X represents a halogen atom for acid halides and OCOR for anhydrides. The examples of the acylation reagent include acetyl chloride or bromide, propionyl chloride or bromide, benzoyl chloride or bromide, adipoyl chloride or bromide, acryloyl chloride or bromide, 2-bromoisobutyryl chloride or bromide, acetic anhydride, benzoyl anhydride and the like.

Acid halides are one of a large group of organic substances containing the halocarbonyl group, being represented by the general formula RCO—X, wherein X represents a halogen atom (fluorine, chlorine, bromine, and iodine) and R represents a group selected from the group consisting of an aliphatic group, an alicyclic group, an aromatic group, hydrogen atom and the like. The terms acyl and aroyl halides refer to aliphatic or aromatic derivatives, respectively. An acid halide may be prepared by replacing —OH group of a carboxylic acid with chlorine atom with heating in the presence of PCl₅, PCl₃ or SOCl₂. An carboxylic acid (X=OH) itself can function as an acylating agent when it is protonated by a strong acid catalyst as in the direct esterification of an alcohol. The most important acid halides are acyl chlorides because they are more easily prepared, more stable and less expensive.

Acid anhydrides are organic compounds that have two acyl groups bound to the same oxygen atom. Most commonly, the acyl groups are derived from the same carboxylic acid, with the formula of the anhydride being (RCO)₂O. Mixed (or unsymmetrical) acid anhydrides, such as acetic formic anhydride, are known. One or both acyl groups of an acid anhydride may also be derived from a sulfonic acid or a phosphonic acid.

In this specification, the chloroformate may be an aryl or alkyl ester of chloroformic acid. The examples of the chloroformate include phenylchloroformate, benzylchloroformate, fluorenylmethyloxycarbonylchloride and the like.

In this specification, the organic acids are organic compounds with acidic properties. The most common organic acids are carboxylic acids, and sulfonic acids, containing the group —SO₃H.

In this specification, the sulfonic acids usually refer to a member of the class of organic acids with the general formula R—SO₃H, wherein R usually represents a hydrocarbon group or an aryl group. The examples of the sulfonic acid include methanesulfonic acid, p-toluenesulfonic acid and the like.

In addition, the above-mentioned aryl group optionally has, on the ring, 1 to 3 substituents selected from a halogen (chlorine, bromine, iodine, fluorine) atom, a C₁-C₆ alkyl group (e.g., an alkyl group having 1 to 6 carbon atoms such as methyl group, ethyl group, propyl group, isopropyl group, butyl group, secondary butyl group, tertiary butyl group and the like), a C₁-C₆ alkoxy group (e.g., an alkoxy group having 1 to 6 carbon atoms such as methoxy group, ethoxy group, propoxy group, isopropoxy group, butoxy group, secondary butoxy group, tertiary butoxy group and the like), hydroxyl group, nitro group, amino group, cyano group and an aminoalkoxy group (an alkoxy group substituted by primary, secondary or cyclic amino group), and a 5- or 6-membered heteroaryl group.

In the reaction of a compound represented by formula 1 and an alkali metal salt of hexacyanoferrate(II) or a hydrate thereof in step 1, the reaction temperature may be from 110° C. to 140° C., preferably from 120° C. to 130° C., more preferably about 125° C. By reacting a bromophenyl morpholine compound with an alkali metal salt of hexacyanoferrate(II) or a hydrate thereof in a reaction mixture comprising a Na₂CO₃, an organophosphorus compound, and a palladium catalyst, a cyanophenyl morpholine can be produced at a temperature that is industrially preferable. The reaction may be conducted for 1-48 hours, preferably 3-16 hours.

In the reaction of the compound represented by formula 2 and hydroxylamine or hydroxylamine hydrochloride in step 2, the reaction temperature may be from 10° C. to 40° C., preferably from 20 ° C. to 30° C. more preferably room temperature. The reaction may be conducted for 1-48 hours, preferably 12-36 hours, more preferably about one day. The reaction may be conducted in a suitable solvent such as an alcohol, an ether or a mixture thereof, preferably with stirring.

In the reaction of the compound represented by formula 3 with an acylation reagent in step 3, the reaction temperature is not particularly limited and may be about room temperature. The reaction may be conducted for 0.5 hour to 5 days, preferably one hour to 4 days. The reaction may be conducted in a suitable solvent such as an amide, an ether, an hydrocarbon or a mixture thereof, preferably with stirring.

In the reaction of the compound represented by formula 4 in step 4, the reaction temperature may be from 60° C. to 140° C., preferably from 80° C. to 120° C., more preferably from 90° C. to 115 ° C. The reaction mixture after the reaction of step 3 without any purification and treatment may be kept at the above temperature. The reaction may be conducted for 1-15 hours, preferably 3-10 hours.

### EXAMPLES

The present invention is explained more specifically with reference to examples. However, the scope of the present invention is not limited to the following examples.

Compound I can be manufactured from the route of Scheme 2 shown below.

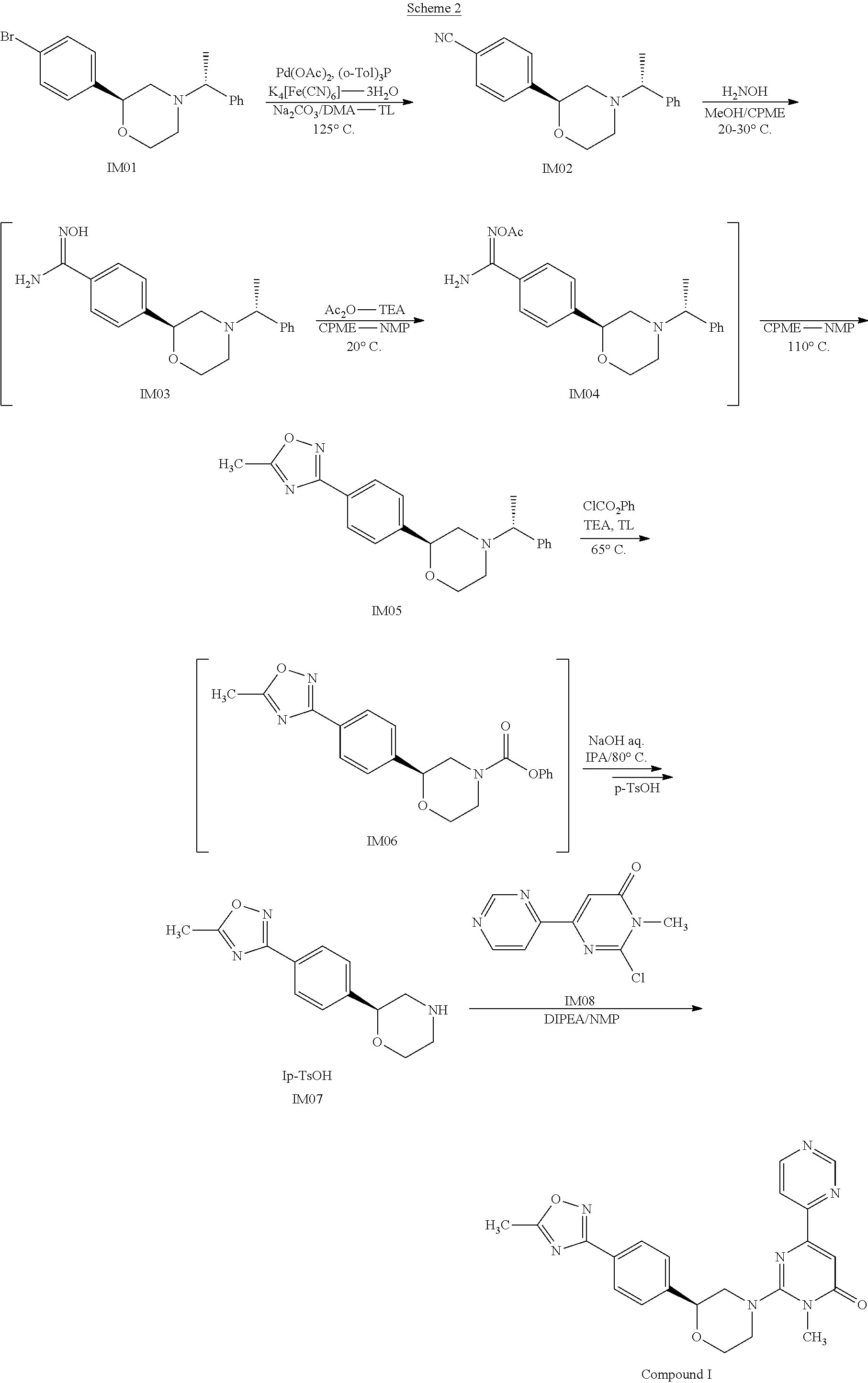

### Abbreviation:

Pd(OAc)₂: Palladium(II) acetate; (o-Tol)₃P: Tris(o-toluoyl)phosphine;
p-TsOH: p-Toluenesulfonic acid; DMA: N,N-dimethylforumamide; TL: Toluene;
CPME: cyclopentylmethylether; NMP: N-methylpyrroridone; TEA: Triethylamine;

### IPA: Isopropylalchol; DIPEA: Diisopropylethylamine

### Reference Example 1

### 2-Chloro-1-methyl-1H-[4,4′]bipyrimidinyl-6-one (IM08)

A suspension of 2-mercapto-1-methyl-1H-[4,4′]bipyrimidinyl-6-one (8.8 g, 40 mmol) in dimethylformamide (30 ml) and 1,2-dichloroethane (30 ml) was added to phosphorus oxychloride (11.2 ml, 120 mmol), and the mixture was stirred at 65° C. for 50 minutes. The solution was poured into ice-cooled dichloromethane (300 ml), and the mixture was added with water and stirred vigorously for 5 minutes. Aqueous sodium carbonate solution (25.4 g, 240 mmol, in water (100 ml)) was added to the mixture and the pH was adjusted to 8 with saturated aqueous sodium hydrogen carbonate solution. Aqueous sodium hypochlorite solution (5% in water, 120 ml) was then added to the mixture. After the mixture was filtered with celite, the organic layer was separated and the aqueous layer was extracted with dichloromethane twice. The organic layers were combined and washed with saturated aqueous sodium hydrogen carbonate solution and dried over sodium sulfate. The solvent was removed under reduced pressure, the residue was purified by silica gel column chromatography (eluent; ethyl acetate/hexane=1/1) and washed with diethyl ether to give 2-chloro-1-methyl-1H-[4,4′]bipyrimidinyl-6-one (2.2 g, 62%, 98.7% purity) as a pale-yellow solid.

¹H-NMR (CDCl₃) δ; 3.74(3H,s), 7.58(1H,s), 8.19(1H, d, J=5.7 Hz), 8.92(1H, d, J=5.2 Hz), 9.31(1H, d, J=1.1 Hz)

An optically active 2-(4-bromophenyl)morpholine (IM01) can be prepared via an optically active 2-ethanolamine compound as described in WO 2007/011065. Synthesis of (S)-2-(4-bromophenyl)-4-(R)-1-phenylethyl)morpholine (IM01) can also be prepared by the following procedure.

### Example 1

### Synthesis of (S)(R)-1-phenylethylamino)-1-(4-bromophenyl)ethanol

(1) A solution of 400 g of (+)-B-chlorodiisopinocamphenylborane (DIP-chloride, 1.25 mol) in THF (400 mL) was added to a solution of 4-bromophenacyl bromide (278 g, 1 mol) in THF (2000 mL) at 0-10° C. After stirring for 2 hours, the solution was allowed to warm to room temperature and the mixture was concentrated and added with TBME (1 L) and 160 g of diethanolamine at 5-10° C. After stirring for one hour, the reaction mixture was filtered and the filtrate was extracted with n-Heptane (1 L) and TBME (1 L). To the combined mixture, water (200 mL) and 8N-NaOH (200 mL) were added and the resulting mixture was stirred for 2 hours, added with 8N-NaOH (50 mL), and stirred for 4 hours. The organic layer was collected, washed with water (400 mL), and concentrated. (R)-1-Phenylethylamine (180 mL, 1.41 mol) was added to the mixture and the resulting mixture was warmed to 130° C. and stirred for 3 hours. The mixture was cooled to 50° C. and added with n-Heptane (500 mL). The mixture was kept for 12 hours and added with n-Heptane (500 mL). The mixture was warmed to 80° C. and then cooled to 5° C. The precipitate was collected by filtration, washed with n-Heptane (150 mL) and dried to give (S)(R)-1-phenylethylamino)-1-(4-bromophenyl)ethanol (195.5 g) as a white solid (yield 61% for 3 steps).

¹H-NMR(CDCl₃) δ: 1.40 (3H, m), 2.50 (1H, m), 2.78 (1H, m), 3.84 (1H, m), 4.68 (1H, m), 7.15 (1H, m), 7.35 (1H, m), 7.43 (1H, m).

(2) (S)-2-(4-bromophenyl)-4-(R)-1-phenylethyl)morpholine was synthesized by a procedure described in WO 2008/078837.

### Example 2

### Synthesis of 4-((S)-4-((R)-1-phenylethyl)morpholine-2-yl) benzonitrile (IM02)

A solution of 1.30 g of Pd(OAc)₂ (5.78 mmol) and 1.76 g of P(o-tolyl)₃ (5.78 mmol) in DMA (200 mL) was added to a suspension of 100 g of (S)-2-(4-bromophenyl)-4- (R)-1-phenylethyl)morpholine (IM01, 288.8 mmol), 48.8 g of potassium hexacyanoferrate(II) (115.52 mmol), 30.6 g of Na₂CO₃ (288.8 mmol) in toluene (200 mL) and DMA (200 mL) at room temperature. The suspension was warmed to 125° C. and stirred for 4 hours. After cooling the mixture, the reaction mixture was filtered and washed with toluene (200 mL X 2). The filtrate was washed with 400 mL water, the aqueous layer was extracted with 200 mL of toluene, and the combined organic layer was washed with 200 mL of water. The organic layer was evaporated and added with 600 mL of isopropanol, and the resulting mixture was concentrated to 300 mL. After the addition of 900 mL of isopropanol, the mixture was added with 10 g of activated charcoal at 60° C. and filtered with celite. The filtrate was washed with 200 mL of isopropanol, evaporated to about 500 mL, and added with 500 mL of H₂O. The resulting slurry was filtered and washed with 200 mL of isopropanol/H₂O (1/1) and dried at 60° C. to give 4-((S)-4-((R)-1-phenylethyl)morpholine-2-yl)benzonitrile (IM02, 73.20 g) as a white solid (yield 87%).

¹H-NMR(CDCl₃) δ: 1.40 (3H, m), 2.02 (1H, m), 2.14 (1H, m), 2.65 (1H, m), 3.08 (1H, m), 3.38 (1H, m), 3.74 (1H, m), 3.92 (1H, m), 4.65 (1H, m), 7.15-7.40 (5H, m), 7.47 (2H, m), 7.56 (2H, m).

### Example 3

### Synthesis of (S)-2-(4-(5-methyl-1,2,4-oxadiazol-3-yl)phenyl)-4-((R) -1-phenylethyl) morpholine (IM05)

Hydroxylamine (5.65 g, 50% in water, 85.5 mmol) was added to a solution of 10 g of 4-((S)-4-((R)-1-phenylethyl)morpholine-2-yl)benzonitrile (34.2 mmol) in cyclopentyl methyl ether (30 mL) and methanol (50 mL) at room temperature and the resulting mixture was stirred for one day. The mixture was added with cyclopentyl methyl ether (70 mL) and 10% NaCl solution (in water). The resulting organic layer was added with N-methylpyrrolidone (20 mL). Solvent was evaporated to obtain 180 mL of residual solution. The solution was added with cyclopentyl methyl ether (50 mL) and evaporated to 130 mL to give a solution of N′-hydroxy-4-((S)-4-((R)-1-phenylethyl) morpholine-2-yl)benzamidine (IM03). A part of the compound was isolated and characterized.

¹H-NMR (CDCl₃) δ: 8.01 (1H, br), 7.61 (2H, d, J=8.3 Hz), 7.41 (2H, d, J=8.3 Hz), 7.35-7.16 (5H, m), 4.86 (2H, br), 4.64 (1 H, dd, J=10.0, 2.4 Hz), 3.93 (1H, ddd, J=11.4, 1.5, 1.5 Hz), 3.75 (1H, ddd, J=11.5, 11.4, 2.4 Hz), 3.37 (1H, q, J=6.6 Hz), 3.12 (1H, m), 2.61 (1H, m), 2.14 (1H, ddd, J=11.5, 11.0, 1.5 Hz), 2.07 (1H, ddd, J=11.0, 2.4, 1.5 Hz), 1.37 (3H, d, J=6.6 Hz):

Triethylamine (4.16 g , 41.04 mmol) and Ac₂O (3.84 g, 37.62 mmol) were added to the above solution at room temperature. After stirring for 3 days, IM04 was obtained and the mixture was warmed to 100° C. and stirred for 7 hours. Aqueous solution (80 mL) of KHCO₃ (10%) was added to the mixture and organic layer was extracted. The mixture was washed with 50 mL of water and the organic layer was evaporated to about half volume. n-Heptane (80 mL) was added and the mixture was cooled to 3° C. The precipitate was filtered, washed with n-Heptane and dried to give 2-(4-(5-methyl-1,2,4-oxadiazol-3-yl) phenyl)-4-((R)-1-phenylethyl)morpholine (IM05, 10.50 g, 87.9%) as a white solid.

¹H-NMR(CDCl₃) δ: 1.37 (3H, m), 2.12 (2H, m), 2.62 (1H, m), 2.63 (3H, s), 3.08 (1H, m), 3.14 (1 H, m), 3.37 (1 H, m), 3.77 (1 H, m), 3.93 (1 H, m), 4.66 (1 H, m), 7.28 (4H, m), 7.49 (2H, m), 8.04 (2H, m).

### Example 4

### Synthesis of (S)-2-(4-(5-methyl-1,2,4-oxadiazol-3-yl)phenyl)morpholine p-toluenesulfonate

A solution of phenyl chloroformate (8.96 g, 57.236 mmol) in THF (10 mL) was added to a solution of (S)-2-(4-(5-methyl-1,2,4-oxadiazol-3-yl)phenyl)-4-((R)-1-phenylethyl)morpholine (IM05, 10.0 g, 28.618 mmol) in THF (50 mL) at 68-69° C. The mixture was stirred for 3.5 hours and triethylamine (0.80 mL, 5.724 mmol) in THF (10 mL) was added to the mixture. After stirring for one hour, the mixture was cooled and concentrated. Isopropanol (100 mL) was added to the mixture and the volume was adjusted to 30 mL by evaporation to give IM06 in isopropanol. A part of the compound was isolated and characterized.

NMR (CDCl₃) δ: 8.08 (2H, d, J=8.2 Hz), 7.53 (2H, d, J=7.6 Hz), 7.38 (2H, dd, J=8.2, 7.6 Hz), 7.22 (1H, dd, 7.6 Hz), 7.14 (2H, d, J=7.6 Hz), 4.61 (1H, dd, J=10.7, 2.7 Hz), 4.32 (1H, br), 4.20 (1H, m), 4.15 (1H, dd, J=11.7, 2.2 Hz), 3.83 (1H, ddd, J =12.0, 11.7, 2.7 Hz), 3.35-2.94 (2H, m), 2.66 (3H, s).

A solution of 25% NaOH in water (28.5 mL) and water (28.5 mL) was added to the IM06 solution and the mixture was warmed to 80° C. and stirred for 3 hours. The resulting organic layer was separated and aqueous layer was extracted with isopropyl acetate (50 mL). The combined organic layer was evaporated and isopropyl acetate (70 mL) was added to the residue. The solution was washed with a mixed solution of 8N-NaOH in water (10 mL) and water (40 mL), the aqueous layer was extracted with isopropyl acetate (50 mL). The combined organic layer was evaporated and isopropyl acetate (70 mL) was added to the residue. p-Toluenesulfonic acid (5.44 g, 28.62 mmol) in THF (25 mL) was added and the mixture was warmed to 45° C. and the mixture was aged for one hour and cooled to room temperature, and filtered. The filtrate was washed with isopropyl acetate (30 mL) and dried to give (S)-2-(4-(5-methyl-1,2,4-oxadiazol-3-yl)phenyl)morpholine p-toluenesulfonate (IM07, 11.41 g, 95.6%) as white solid.

¹-NMR(DMSO-d6) δ: 2.36 (3H, s), 2.65 (3H, s), 3.08 (1H, m), 2.63 (3H, s), 3.08 (1H, m), 3.37 (4H, m), 3.50 (1H, m), 4.00 (1H, m), 4.25 (1H, m), 7.23 (1H, m), 7.55 (1H, m), 7.71 (1H, m), 8.06 (1H, m).

### Example 5

### 2-{(2S)-2-[4-(5-Methyl-[1,2,4]oxadiazol-3-yl)phenyl]morpholin-4-yl}-1-methyl-1H-[4,4′]bipyrimidinyl-6-one (Compound I)

N,N′-Diisopropylethylamine (2.23 g, 17.25 mmol) was added to the mixture of (S)-2-(4-(5-methyl-1,2,4-oxadiazol-3-yl) phenyl) morpholine p-toluenesulfonate (IM07, 3.00 g, 7.19 mmol) and 2-chloro-1-methyl-1H-[4,4′]bipyrimidyl-6-one (IM08, 1.568 g, 7.04 mmol) in NMP (15 mL) at 55° C. over 60 minutes. The mixture was added with water (22.5 mL) and cooled, and the precipitate was filtered and washed with water (15 mL), and dried to give 2-{(2S)-2-[4-(5-Methyl-[1,2,4]oxadiazol-3-yl)phenyl]morpholin-4-yl}-1-1-methyl-1H-[4,4′]bipyrimidinyl-6-one (Compound I, 2.95 g, 97.1%) as pale-brown solid.

¹H-NMR(CDCI₃) δ: 3.09 (dd, J=12.9, 10.8 Hz, 1H), 3.29 (m, 1H), 3.52-3. 64 (m, 2H), 3.59 (s, 3H), 4.00 (m, 1H), 4.21 (m, 1H), 4.72 (dd, J=10.5, 2.1 Hz, 1H), 7.07-7. 13 (m, 2H), 7.38-7.43 (m, 3H), 8.13 (dd, J=5.4,1. 2 Hz, 1H), 8.88 (d, J=5.1 Hz, 1H), 9.28 (s, 1H).

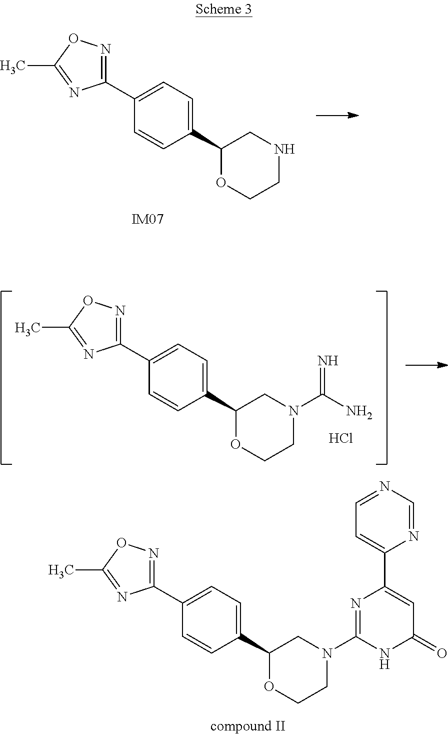

### Example 6

### 2-{(2S)-2-[4-(5-Methyl-[1,2,4]oxadiazol-3-yl)phenyl]morpholin-4-yl}-1H-[4,4′]bipyrimidinyl-6-one (Compound II)

To a solution of (2S)-2-(4-(5-methyl-1,2,4-oxadiazol-3-yl)phenyl)morpholine hydrochloride (IM07: 4.00 g, 14.2 mmol) and 1H-pyrazole-1-carboxamidine hydrochloride (2.19 g, 14.9 mmol) in N,N-dimethylformamide (14 ml ) was added N,N-diisopropylethylamine (4.05 g, 31.3 mmol) at room temperature and the solution was stirred for 4 hours. The solution was decanted with ether, then 3-oxo-3-pyrimidin-4-yl-propionic acid ethyl ester (3.59 g, 18.5 mmol), the resulting solution was added with potassium carbonate (4.92 g, 35.6 mmol) and ethanol (30 ml ). After refluxed for 18 hours, the solution was concentrated under reduced pressure. The residue was washed with water and the hot mixture of ethanol and 1N hydrochloric acid(1/1, v/v) , and dried under reduced pressure to afford 2-{(2S)-2-[4-(5-methyl-[1,2,4]oxadiazol-3-yl)-phenyl]-morpholin-4-yl}-1H-[4,4′]bipyrimidinyl-6-one (Compound II, 1.94 g, 33%).

### INDUSTRIAL APPLICABILITY

Since the compound obtained according to the production method of the present invention can be an important starting material for synthesizing 2-(2-(4-oxadiazolylphenyl)morpholin-4-yl)-1-methyl-1H-[4,4′]bipyrimidinyl-6-one, which is useful as a therapeutic drug for Alzheimer' s disease and the like, the present invention can contribute to the provision of a useful pharmaceutical agent.

1. A method for preparing an optically active 2-{4-(5-substituted-oxadiazolyl) phenyl}morpholine represented by formula 5, which comprises the following steps 1) to 4): 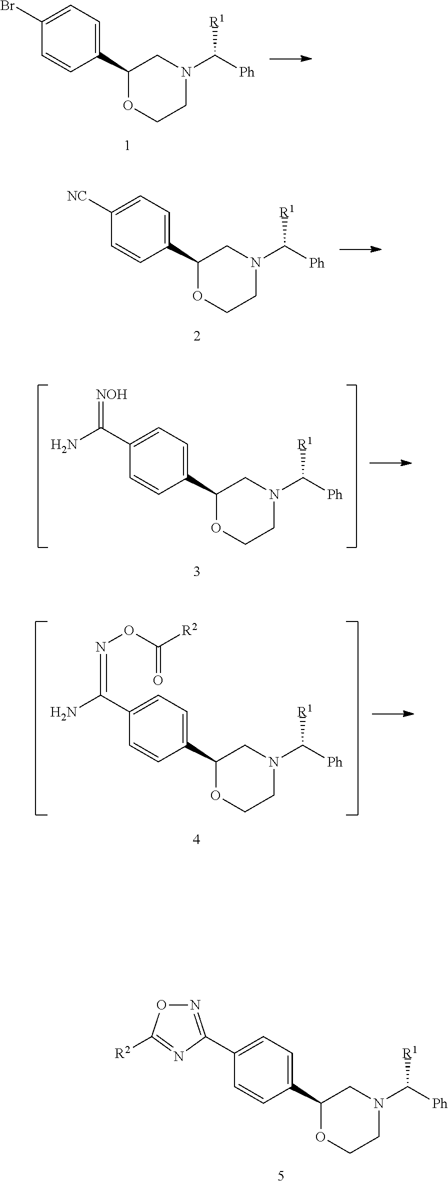
wherein R¹ represents hydrogen atom or a C₁-C₆ alkyl group, R² represents a C₁-C₆ alkyl group, a substituted C ₁-C₆ alkyl group, or an aryl group,
1) reacting a compound represented by formula 1 with an alkali metal salt of hexacyanoferrate(II) or a hydrate thereof at a temperature of from 110° C. to 140° C. in a reaction mixture comprising a Na₂CO₃, an organophosphorus compound, and a palladium catalyst in a polar aprotic solvent alone or combination of a polar aprotic solvent and other polar aprotic solvent or hydrocarbon solvent to give a compound represented by formula 2,
2) reacting the compound represented by formula 2 with hydroxylamine or hydroxylamine hydrochloride at a temperature of from 10° C. to 40° C. in an aprotic polar solvent to give a compound represented by formula 3,
3) reacting the compound represented by formula 3 with an acylation reagent selected from the group consisting of aliphatic acyl halides, aromatic acyl halides, aliphatic acyl anhydrides and aromatic acyl anhydrides, and
4) keeping the mixture obtained after step 3) at a temperature of from 60° C. to 140° C. to give a compound represented by formula 5.

2. The method of claim 1, wherein the organophosphorus compound is 2-dicyclohexylphosphino-2′,4′,6′-triisopropylbiphenyl, 1,1′-bis(diphenylphosphino) ferrocene, tri(o-tolyl)phosphine, or 1,3-bis(diphenylphosphino)propane; the polar aprotic solvent is N,N-dimethylacetoamide, N-methylpyrroridone, N,N-Dimethylformamide, or tetrahydrofuran; and the combination of an polar aprotic solvent and other polar aprotic solvent or hydrocarbon solvent is N,N-dimethylacetoamide-toluene.

3. The method of claim 1, wherein R¹ is methyl group; R² is a phenyl group having 1 to 3 substituents selected from the group consisting of a halogen atom, a C₁-C₆ alkyl group, and a C₁-C₆ alkoxy group.

4. A method for preparing optically active 2-{4-(5-substituted-oxadiazolyl)phenyl}morpholines represented by the formula 7, which comprises the following steps 5) and 6): 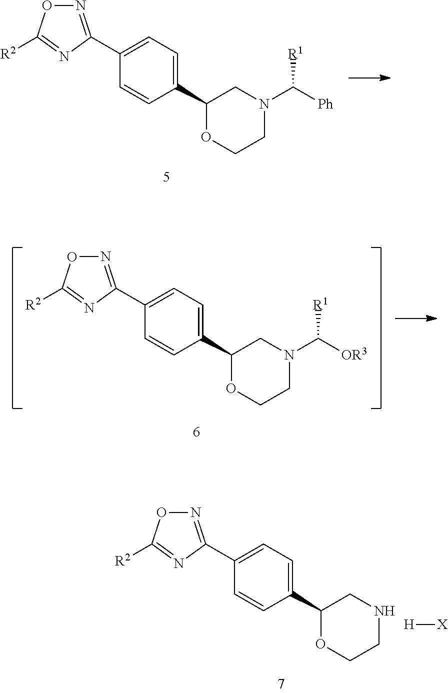
wherein R¹ represents hydrogen atom or a C₁-C₆ alkyl group; R² represents a C ₁-C₆ alkyl group, a C₆-C₁₀ aryl group, or a 5- or 6-membered heteroaryl group; R³ represents a phenyl group, a benzyl group, or a fluorenylmethyl group; H—X represents an organic acid,
5) reacting the compound represented by formula 5 with a chloroformate selected from the group consisting of phenylchloroformate, benzylchloroformate, and fluorenylmethyloxycarbonylchloride in a solvent to give a compound represented by formula 6, and
6) subjecting the compound represented by formula 6 to hydrolysis or aminolysis, and reacting the resulting unprotected 2-{4-(5-substituted-oxadiazolyl)phenyl}morpholine with an organic acid in a polar solvent to give the compound represented by formula 7.

5. The method of claim 4, wherein R² is methyl group; R³ is phenyl group.

6. An optically active 2-{4-(5-substituted-oxadiazolyl)phenyl}morpholine represented by formula 8: 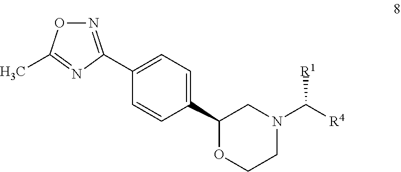
wherein R¹ represents hydrogen atom or a C₁-C₆ alkyl group; R⁴ represents a C₁-C₆ alkyl group or an aryl group optionally having substituent(s).

7. The compound of claim 6, wherein R¹ is methyl group; R⁴ is a phenyl group having 1 to 3 substituents selected from the group consisting of a halogen atom, a C₁-C₆ alkyl group, a substituted C₁-C₆ alkyl group, and a C₁-C₆ alkoxy group.

8. An optically active compound represented by formula 9: 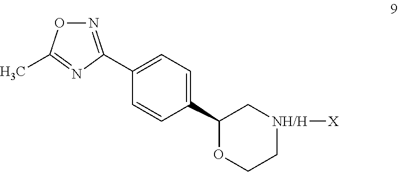
wherein H—X represents a C₁-C₅ alkyl carboxylic acid, a C₁-C₅ alkyl sulfonic acid, an aryl-carboxylic acid, or an aryl sulfonic acid,
wherein the aryl group optionally having 1 to 3 substituent(s) selected from the group consisting of a halogen atom, a C₁-C₆ alkyl group, a substituted C₁-C₆ alkyl group, and a C₁-C₆ alkoxy group.

9. The compound of claim 8, wherein H—X is a p-toluene sulfonic acid.

10. A method for preparing an optically active compound (I), 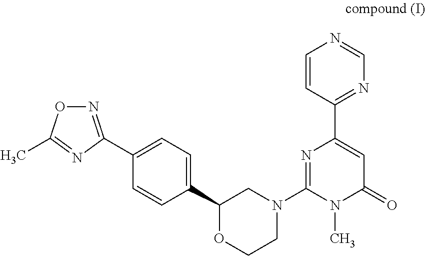
comprising the method of claim 1.

11. A method for preparing active compound (I), 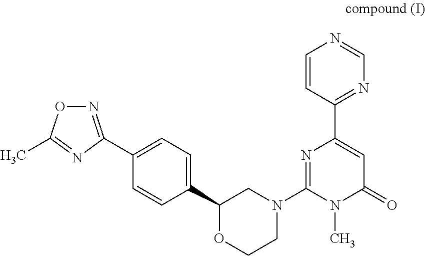
comprising the method of claim 4.
